(19) United States
(12) Patent Application Publication
(10) Pub. No.: US 2015/0223824 A1
(43) Pub. Date: Aug. 13, 2015

(54) JIG WITH TARGETING FEATURE

(71) Applicant: Biomet Manufacturing, LLC., Warsaw, IN (US)

(72) Inventor: Edward Mebarak, Miami, FL (US)

(73) Assignee: Biomet Manufacturing, LLC., Warsaw, IN (US)

(21) Appl. No.: 14/178,921

(22) Filed: Feb. 12, 2014

(51) Int. Cl. A61B 17/17

(52) U.S. Cl. CPC A61B 17/17

(57) ABSTRACT

The present invention relates to a surgical jig incorporating a targeting feature. The surgical jig includes a jig body with a first end, a second end, an anchoring opening, and a neck portion at the first end of the jig body. The neck portion allows the surgical jig to releasably attach to an anchor such as a bone plate. A guiding body with at least one guide opening formed within is attached to the jig body at the jig body's second end. A targeting feature is fixedly associated with the guide opening of the guiding body and can include a first region and a second region with the first region having a different imaging property than the second region.

### BACKGROUND OF THE INVENTION

1. Field of the Invention

The present invention relates to a jig, and, more particularly, to a surgical jig.

2. Description of the Related Art

Jigs are often used during surgery to align devices to be implanted with features or other devices that are already in the body. The jig allows the surgeon to align an implant to the proper site without the need to fully expose the implantation site during surgery. Combining the use of a jig with an imaging technique, such as fluoroscopy, allows the surgeon to make a smaller incision around the implantation site which reduces the risk of infection and recovery time for the patient.

Jigs are known in the art that align guide holes in the jig's guiding body with an implant's target site. Such jigs typically have a portion that connects the jig to an anchor within the body such that the guide holes in the guiding body are aligned with a target. Once the jig is anchored, the surgeon need only put the implant, such as a bone screw, through the corresponding guide hole in the jig body to align the implant with its target.

One problem with the simple kinds of jig described above is that it can be difficult to know the exact position of where the implant will end up relative to the body structures around the implant site. For example, forces or torques applied to the jig during surgery can alter the alignment of the jig's guide holes with the target or the target may be located a substantial distance from the incision. To address this problem, jigs have been developed to incorporate targeting features that give the surgeon a better estimate of where an implant will ultimately reside once implanted. An example of such a jig is described by U.S. Pat. No. 8,241,286 (Metzinger et al.) which is incorporated herein. Metzinger et al. describes a jig with a target hole through which a target wire can be inserted. The jig can connect to a nose component that includes an alignment sight made from a radio-opaque material. When the surgeon positions the target wire through the target hole, the image of the target wire can be radioscopically viewed along a line of sight to bisect the image of the alignment sight. This gives the surgeon a visual reference under fluoroscopy to better determine where the lag screws will be placed in the bone. While the jig of Metzinger et al. is an improvement to its predecessors, its most practical use is to show alignment of a guide pin relative to a patient's femoral neck.

What is needed in the art is a targeting feature for surgical jigs that is simple and allows for proper visual alignment of a target and surgical jig.

### SUMMARY OF THE INVENTION

The present invention provides a surgical jig that incorporates a targeting feature.

The invention in one form is directed to a surgical jig including a jig body, a guiding body, and at least one targeting feature. The jig body has a first end, a second end, and a neck portion at the first end of the jig body. The neck portion of the jig body is configured to allow the surgical jig to be releasably attached to an anchor, such as a bone plate. The guiding body is connected to the jig body at the jig body's second end. The guiding body has at least one guide opening formed in the guiding body along a guide axis. The guide opening has a center defining an alignment axis. At least one targeting feature is in fixed association with a guide opening of the guiding body. The targeting feature can partially surround its associated guide opening and have a first region and a second region. The targeting feature can have a first region with a different imaging property, such as material composition or density, than its second region.

The invention in another form is directed to a surgical jig including a jig body, a guiding body, a targeting sight, and at least one targeting feature. The jig body has a first end, a second end, and a neck portion at the first end of the jig body. The neck portion of the jig body is configured to allow the surgical jig to be releasably attached to an anchor, such as a bone plate. The guiding body is connected to the jig body at the jig body's second end. The guiding body has at least one guide opening formed in the guiding body along a guide axis. The guide opening has a center defining an alignment axis. The targeting sight is in fixed association with the guiding body and has at least one targeting feature associated with the targeting sight. The targeting feature can have a first region and a second region, with the first region having a different imaging property than the second region. The targeting feature can be formed within the targeting sight.

The invention in another form is directed to a method for aligning a surgical jig with a target. A surgical jig is provided that has at least one guide opening defining a guide axis and a center of the guide opening defining an alignment axis. At least one targeting feature is provided that has a first region and a second region, with the targeting feature defining a targeting axis. The first region of the targeting feature has a different imaging property than the second region. The surgical jig is anchored to a surgical area of a patient. A target of a patient defining a target axis is identified. An area around the target is imaged with a known technique such as fluoroscopy. The targeting feature is positioned so that the targeting axis is simultaneously parallel with both the alignment axis of the guide opening center and the target axis of the target. The surgical jig is positioned so that the guide axis of the guide opening is perpendicular to the target axis of the target. Correct positioning of the surgical jig is verified by the presence of an image of a part of the first region and an image of a part of the second region.

An advantage of the present invention is that it provides a relatively simple and inexpensive way for a surgeon to align a surgical jig with a target inside a patient using known imaging techniques.

Another advantage is the present invention gives the physician a reference location of the surgical jig to the target, allowing the physician to properly align the surgical jig more quickly and with fewer images. Fewer images reduce costs and, when fluoroscopy is used, results in less potentially harmful radiation exposure to the surgical team and patient.

### BRIEF DESCRIPTION OF THE DRAWINGS

The above-mentioned and other features and advantages of this invention, and the manner of attaining them, will become more apparent and the invention will be better understood by reference to the following description of embodiments of the invention taken in conjunction with the accompanying drawings, wherein:

FIG. 1 is a perspective view of an embodiment of the present invention;

FIG. 2 is a top view of a targeting sight of the present invention;

FIG. 3 is a perspective view of another embodiment of the present invention;

FIG. 4 is a depiction of a correct positioning of the present invention when viewed under an imaging technique; and

FIG. 5 is a depiction of an incorrect positioning of the present invention when viewed under an imaging technique.

Corresponding reference characters indicate corresponding parts throughout the several views. The exemplifications set out herein illustrate embodiments of the invention and such exemplifications are not to be construed as limiting the scope of the invention in any manner.

### DETAILED DESCRIPTION OF THE INVENTION

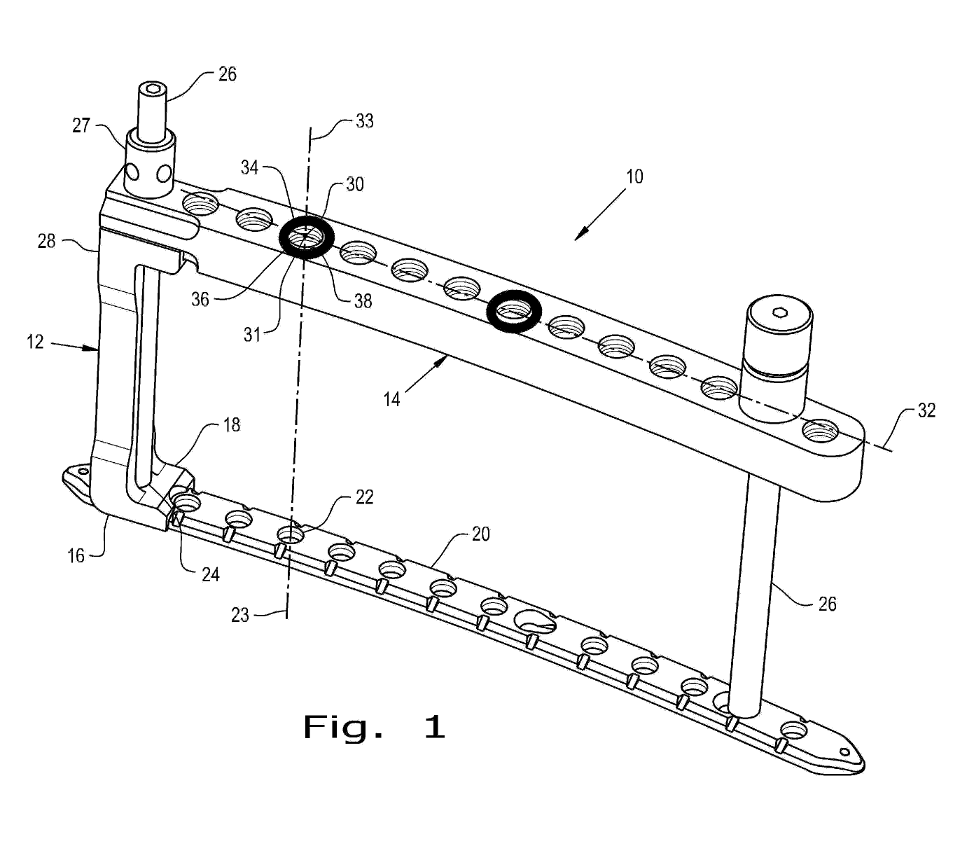

Referring now to the drawings, and more particularly to FIG. 1, there is shown a surgical jig 10 which generally includes a jig body 12 and a guiding body 14 with a targeting feature 34 fixedly attached. The jig body 12 includes a first end 16 which has a neck portion 18 that can be formed as an integral part of the jig body 12 or connected to the jig body 12. Although FIG. 1 shows the jig body 12 as having a vertical portion with two horizontal portions that are perpendicular to the vertical portion, the shape of the jig body 12 can be altered without straying from the present invention.

The neck portion 18 can be releasably attached to an implant or anchor 20, shown as a bone plate, which holds the surgical jig 10 in a mostly fixed position relative to a target 22. The target 22 can be, for example, an attachment opening in an orthopedic implant or a point of the patient's anatomy to which an orthopedic implant, bone plate, etc. is being installed. The neck portion 18 can sit flush with the anchor 20 in order to provide a more stable connection between the jig body 12 and the anchor 20, but it will be appreciated that it is not required for the neck portion 18 to sit flush with the anchor 20. In some cases, it may be desirable for the jig body 12 to have a less static connection with the anchor 20 to allow for easier adjustment of the guiding body 14 relative to the target 22. The neck portion 18 has an anchoring hole 24 that corresponds to a hole (not shown) in the anchor 20 through which an anchoring rod 26 is disposed to connect the jig body 12 to the anchor 20. This type of connection is only exemplary of the many ways that the neck portion 18 can connect the jig body 12 to the anchor 20. The jig body 12 and the neck portion 18 should preferably be composed of materials that are safe to use in a surgical environment where biological fluids are encountered, such as biocompatible polymers, ceramics and metals. Examples of such materials known in the art include polyphenylsulfone (PPSU), polyethylene, polyether ether ketone (“PEEK”), poly(tetrafluroethylene) (“PTFE”), poly(dimethylsiloxane) (“PDMS”), hydroxyapatite, stainless steel, and titanium. The material(s) chosen to be used in the jig body 12 and neck portion 18 can be selected to provide desirable characteristics, such as re-usability, without straying from the scope of the present invention. Manipulating these materials into a desired shape are well known in the art and include techniques such as machining, molding, extrusion, sintering, and additive manufacturing (“3D printing”).

The guiding body 14 of the surgical jig 10 is connected to the jig body 12 at a second end 28 of the jig body 12. The guiding body 14 is attached to the jig body 12 with the anchoring rod 26 disposed through openings (not shown) formed in the guiding body 14 and the jig body 12. Disposed over the anchoring rod 26 is an anchoring nut 27 to which a torque can be applied to tighten or loosen the connection between the jig body 12 and the guiding body 14. The connection between the jig body 12 and the guiding body 14 can be accomplished using other means, such as a clamp, welding, etc. that don't require alignment of holes formed in the jig body 12 and guiding body 14. The connection between the jig body 12 and the guiding body 14 can be a temporary connection, as shown, or a permanent connection. Optionally, the guiding body 14 can be connected directly to the anchor 20 by another anchoring rod 26 at an end opposite the second end 28 of the jig body 12. Such a connection allows for the guiding body 14 to be more rigidly connected to the anchor 20 and better resist forces and torques that could cause a guide opening 30 on the guiding body 14 to lose its alignment with the target 22.

The guiding body 14 of the surgical jig 10 can be formed to different specifications and shapes depending on the desired use. As shown in FIG. 1, the guiding body 14 has what is commonly referred to as an “arm” shape, extending a comparatively larger length in the longitudinal direction than the transverse direction. Such a configuration is well-suited when there are multiple targets 22 in a long, narrow target area. Other configurations may also be desirable, for example, if there are multiple targets that aren't aligned with each other or if the surgical jig 10 is a part of an apparatus incorporating multiple modular components. In such cases, the configuration of the guiding body 14 can be suitably optimized using techniques known in the art for particular surgical situations without straying from the present invention.

It is desirable, but not necessary, for the material(s) used to form the guiding body 14 to be substantially radio-translucent. As is known in the art, materials viewed under fluoroscopy can create darker or lighter images, depending on an imaging property of the material. As used throughout, “substantially radio-translucent” refers to an imaging property possessed by a material that allows the material to be mostly “seen through” in a fluoroscopic image. “Substantially radio-translucent” is not the same as “completely radio-translucent.” Some materials cast a light grey image, as opposed to a clear or white image, under fluoroscopy that allows a viewer to see darker, more radio-opaque materials through the substantially radio-translucent material. Materials that are considered to be substantially radio-translucent include polymers such as PPSU, polyethylene, PEEK, PTFE, and PDMS. In contrast, “substantially radio-opaque” materials are materials that cast a dark image when viewed under fluoroscopy. The degree of darkness that a substantially radio-opaque material appears as under fluoroscopy varies, but can be described as between grey and black. Materials that are considered to be substantially radio-opaque include metals such as stainless steel, metals with properties similar to stainless steel, and certain ceramics. Comparatively speaking, substantially radio-opaque materials will be visible through substantially radio-translucent materials under fluoroscopy if there is an overlap, with the darkness of the substantially radio-opaque material being the dominant image seen. It is therefore contemplated by the present invention that the term “substantially,” when used to describe imaging properties of materials used in the present invention, can be interchangeable with the term “comparatively,” such that a material can be considered substantially radio-opaque if it is darker, when viewed under fluoroscopy, than a lighter substantially radio-translucent material. Since a material's density is the primary property affecting whether the material is substantially radio-opaque or substantially radio-translucent, it is also contemplated that a substantially radio-opaque material will have a greater density than a substantially radio-translucent material. In addition to being substantially radio-translucent, it is desirable for the material(s) of the guiding body 14 to be biocompatible, able to withstand sterilization using techniques such as autoclaving, and easily manipulated to a variety of different shapes.

The guiding body 14 includes at least one guide opening 30 formed in the transverse direction of the guiding body 14 along a guide axis 32. The guide opening 30 is sized to allow a variety of devices to pass through the guide hole and towards the target 22. Devices that might pass through the guide opening 30 include drill sleeves, k-wires, and bone screws. The guide opening 30 can be threaded, as shown in FIG. 1, or smooth, depending on what devices will be going through the guide opening 30 during use. If more than one guide opening 30 is formed in the guiding body 14, the guide openings 30 can all be aligned along the guide axis 32 or some of the guide openings 30 can be offset from the guide axis 32. Guide openings 30 being offset from the guide axis 32 may be a desirable configuration when there are multiple targets 22 not aligned with each other or when designing the guiding body 14 to be used in many different situations. If the guiding body 14 is configured with guide openings 30 being offset from the guide axis 32, each offset guide opening 30 will have another guide axis (not shown) that intersects the guide axis 32. Each guide opening 30 has a center 31 through which an alignment axis 33 runs. The center 31 can be a geometric center of the guide opening 30 or a point in an area within the guide opening 30. The guide axis 32 and the alignment axis 33 may perpendicularly intersect at the center 31 of the guide opening 30.

In one embodiment of the present invention, the guiding body 14 has at least one targeting feature 34 in fixed association with a guide opening 30. The targeting feature 34 has a variable shape and can include a first region 36 and a second region 38. The first region 36 is shown in FIG. 1 as a dark annular region surrounding the guide opening 30 and the second region 38 is the white space shown between the first region 36 and the guide opening 30. While the targeting feature 34 is shaped as a concentric circle in FIGS. 1-5, the targeting feature can also be shaped as a concentric square, a concentric triangle, a concentric star, a concentric diamond, or other geometric shapes that can include the first region 36 and the second region 38. Such other shapes of the targeting feature 34 might be selected based on user preference, shape of the target, and shape of the guiding body 14, among other reasons. The first region 36 is composed of a material that has a different imaging property than the material of the second region 38. The different imaging property can be any material property that will cause the first region 36 to create a different observed color under imaging than the second region 38. Such imaging properties can be, e.g., a material density or a material composition. In one embodiment, the first region 36 is composed of a substantially radio-opaque object material and the second region 38 is composed of a substantially radio-translucent object material. The materials used to form the first region 36 and the second region 38 can be selected to achieve a contrast between the first region 36 and the second region 38 when viewed under different forms of imaging. The techniques used to form the targeting feature 14 will vary based on what materials are used, but can include molding, casting, 3-D printing, sintering, forming, and machining techniques that are known in the art.

The targeting feature 34 can be reversibly associated with a guide opening 30 or it can be disposed within the guiding body 14. Ways of fixedly associating the targeting feature 34 with the guide opening 30 can include press fitting the targeting feature 34 into a slot (not shown) formed in the guiding body 14, using an adhesive to connect the targeting feature 34 to the guiding body 14, and melting the targeting feature 34 into the guiding body 14. It is not necessary that the entirety of the targeting feature 34 be a completely separable feature from the guiding body 14. For example, the targeting feature 34 can be formed when the first region 36 is fixedly attached to the guiding body 14, with a lack of or a part of the material of the guiding body 14 in between the first region 36 and the alignment axis 33 being the second region 38 of the targeting feature 34. The targeting feature 34 can also be formed when the second region 38 is fixedly attached to the guiding body 14, with a part of the material of the guiding body 14 in between the second region 38 and the alignment axis 33 being the first region 36. One concern when fixedly associating the targeting feature 34 to the guiding body 14 is the presence of blood and other biological fluids during use. The presence of these biological fluids make it necessary that, if the targeting feature 34 is meant to be permanently attached to the guiding body 14 and re-usable, there is a seal between the targeting feature 34 and the guiding body 14 during use that can prevent biological fluid from being trapped between the targeting feature 34 and the guiding body 14 then later transmitted to a different patient. Optionally, the targeting feature 34 can be separable from the guiding body 14 and sterilized separately.

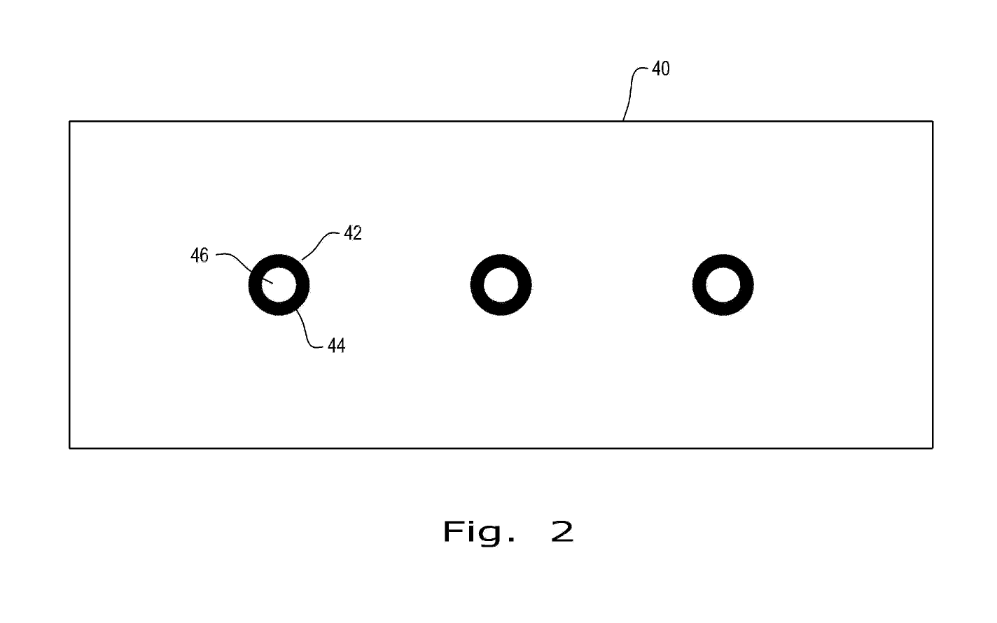

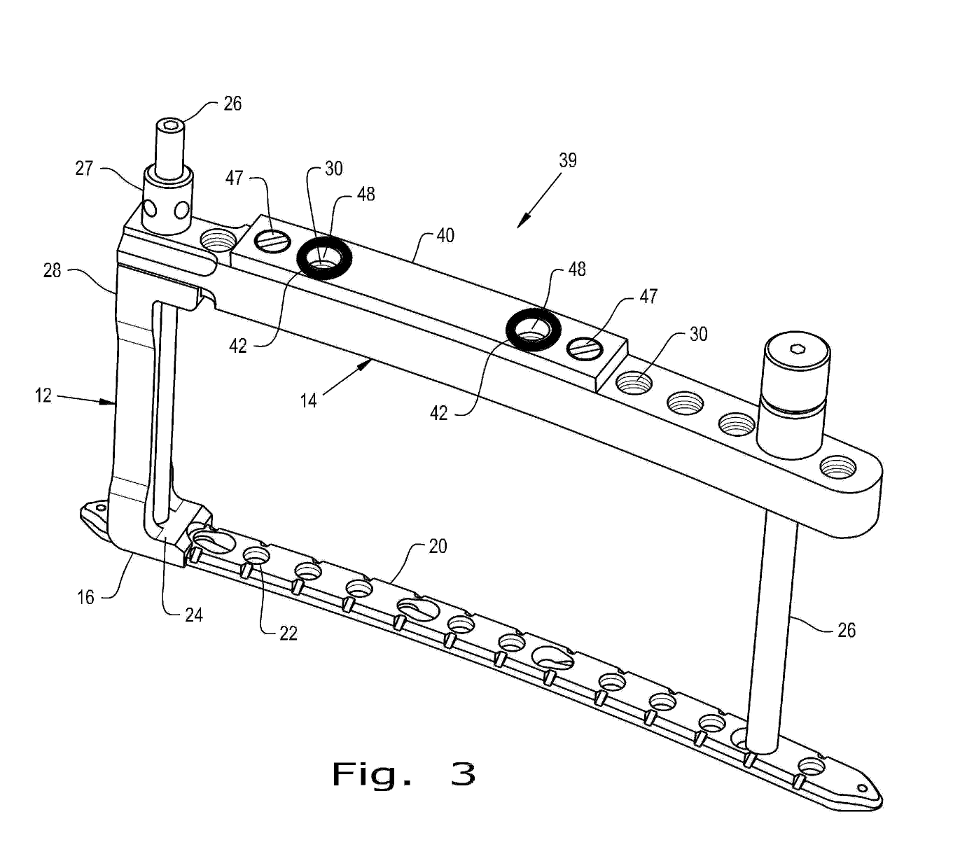

In another embodiment of the present invention, a targeting sight 40 is provided that incorporates a targeting feature 42. As shown in FIGS. 2 and 3, the targeting sight 40 can be a bar of material with the targeting feature 42 formed within the bar. The targeting feature 42 can also be removably attached to the targeting sight 40 similarly to ways of removably attaching the targeting feature 34 to the guiding body 14 described above. The targeting feature 42 can include a first region 44 and a second region 46, with the first region 44 having a different imaging property than the second region 46. In FIGS. 2 and 3, the first region 44 is shown as a darkened annular region surrounding the second region 46 and/or guide opening 30. The first region 44 is at least partially comprised of a substantially radio-opaque material that is visible under imaging. The second region can be comprised of a substantially radio-translucent material that is mostly invisible under imaging or can be a lack of material which will not be visible under imaging. The shape of the targeting feature 42 can vary in a similar manner to the targeting feature 34 described above. The shape of the targeting sight 40 and the arrangement of targeting features 42 arranged thereon can be modified to correspond to different locations of targets and arrangements of surgical jigs that will incorporate the targeting sight 40. The targeting sight 40 can also incorporate ways to alter the location of the targeting feature 42 relative to the targeting sight 40, such as a sliding mechanism.

Referring now to FIG. 3, a surgical jig 39 is shown that includes a jig body 12, a guiding body 14, and a targeting sight 40 with an associated targeting feature 42. The targeting sight 40 is fixedly attached to the guiding body 14 by a pair of screws 47 that correspond with threaded guide openings 30 of the guiding body 14. A pair of sight openings 48 are formed within the targeting sight 40 and are surrounded by targeting features 42 that are fixedly attached to the targeting sight 40. The sight openings 48 are similar in size and shape to their corresponding guide openings 30 to allow for various devices to go through the sight openings 48 during use.

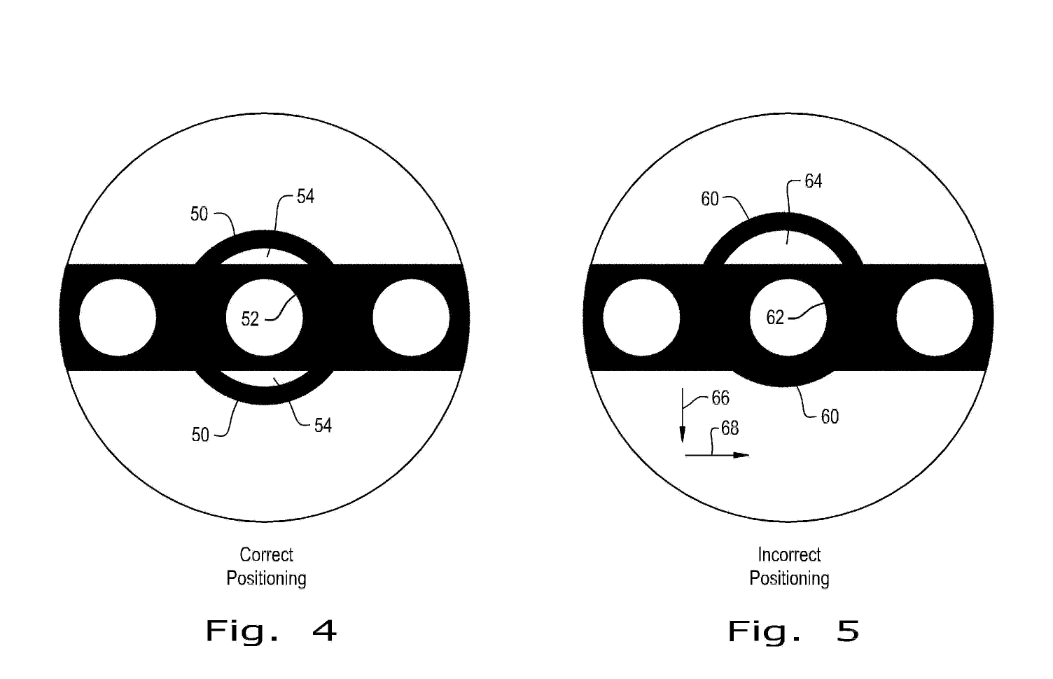

During use, the surgical jig 10, 39 of FIGS. 1 and 3 is fixed to an implant or anchor 20 to allow proper alignment of the guide opening 30 with the target 22. When the guide opening 30 is correctly positioned relative to the target 22, the alignment axis 33 running through the center 31 of the guide opening 30 will overlap with a target axis 23 corresponding to the target 22. When the user believes the guide opening 30 is correctly positioned relative to the target 22, a fluoroscopic image can be taken to compare the alignment of the guide opening 30 with associated targeting feature 34, 42 to the target 22. FIG. 4 shows an example of a fluoroscopic image that would be seen by a user when a guide opening 30 of the present invention is correctly positioned with respect to the target 22. An image of the first region 50 is dark and can be seen symmetrically surrounding an image of the target 52, with an image of the second region 54 being seen as a space between the images of the first region 50 and the target 52. The first region 36, 44 is held in fixed attachment to the guide opening 30 of the guiding body 14, so the image of the first region 50 allows the user to relate the location of the guide opening 30, which is not visible under imaging, to the target 22 by comparing the image of the first region 50 to the image of the target 52.

If the surgical jig 10 is not viewed as positioned correctly under imaging, as shown in FIG. 5, the user is able to determine what adjustments need to be made to the positioning of the guiding body 14 in order to correctly position the guide opening 30 relative to the target 22. If the image shown in FIG. 5 was seen during use, the user would see that an image of the first region 60 is not symmetrically visible about an image of the target 62 and there is no visible image of the second region 64 between the image of the first region 60 and the image of the target 62 below the image of the target 62. To correctly position the guide opening 30 relative to the target 22, the user would move the guiding body 14 with guide opening 30 in directions 66 and 68 sufficiently to obtain correct positioning of the guide opening 30. After adjusting the positioning of the guiding body 14, the user verifies correct positioning of the guide opening 30 when an image similar to that shown in FIG. 4 is obtained. If correct positioning isn't obtained, the user re-positions the guiding body 14 and re-images the surgical jig 10 until an image similar to that shown in FIG. 4 is obtained. It should be appreciated by those skilled in the art that the configuration of the targeting feature 34, 42 will affect what obtained image indicates correct positioning of the surgical jig 10. The targeting feature's shape of the substantially radio-opaque first region and substantially radio-translucent second region and location relative to the guide opening can be altered to obtain a wide variety of images that indicate correct positioning without straying from the present invention.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A surgical jig, comprising:
a jig body having a first end, a second end, at least one anchoring opening, and a neck portion at said first end of said jig body, said neck portion configured to releasably attach said surgical jig with an anchor;
a guiding body connected to said second end of said jig body, said guiding body having at least one guide opening formed in said guiding body along a guide axis and having a center defining an alignment axis; and
at least one targeting feature having a defined geometric position adjacent to said at least one guide opening.

2. The surgical jig according to claim 1, wherein at least a portion of said at least one targeting feature partially surrounds said at least one guide opening.

3. The surgical jig according to claim 2, wherein said at least one targeting feature comprises a first region and a second region, said first region having a different imaging property than said second region.

4. The surgical jig according to claim 3, wherein said different imaging property is at least one of a density or a material composition.

5. The surgical jig according to claim 4, wherein said first region comprises a substantially radio-opaque object material and said second region comprises a substantially radio-translucent object material.

6. The surgical jig according to claim 5, wherein at least a part of said second region is located between at least a part of said first region and said alignment axis.

7. The surgical jig according to claim 5, wherein at least a part of said first region is located between at least a part of said second region and said alignment axis.

8. The surgical jig according to claim 4, wherein said guide axis is perpendicular to a target axis coinciding with a target.

9. The surgical jig according to claim 5, wherein said at least one targeting feature is disposed within said guiding body.

10. The surgical jig according to claim 5, wherein said first region is comprised of at least one of a metal or a ceramic.

11. The surgical jig according to claim 5, wherein a shape of said targeting feature is one of a concentric circle, a concentric square, a concentric triangle, a concentric star, or a concentric diamond.

12. The surgical jig according to claim 5, wherein said anchor is a bone plate.

13. A surgical jig, comprising:
a jig body having a first end, a second end, and a neck portion connected to said first end of said jig body, said neck portion configured to releasably attach said surgical jig with an anchor;
a guiding body formed at said second end of said jig body, said guiding body having at least one guide opening formed in said guiding body along a guide axis and having a center defining an alignment axis;
a targeting sight having a defined geometric position adjacent to said guiding body; and
at least one targeting feature associated with said targeting sight.

14. The surgical jig of claim 13, wherein said targeting feature comprises a first region and a second region, said first region having a different imaging property than said second region.

15. The surgical jig according to claim 14, wherein at least a part of said first region is located between said alignment axis and at least a part of said second region.

16. The surgical jig according to claim 15, wherein a shape of said targeting feature is one of a concentric circle, a concentric square, a concentric triangle, a concentric star, or a concentric diamond.

17. The surgical jig according to claim 15, wherein said at least one targeting feature is disposed within said targeting sight.

18. A method for aligning a surgical jig with a target, the method comprising the steps of:
providing a surgical jig having at least one guide opening defining a guide axis;
providing at least one targeting feature comprising a first region and a second region, said first region having a different imaging property than said second region;
anchoring said surgical jig to a surgical area;
identifying a target within a patient defining a target axis;
imaging an area around said target within a patient;
positioning said surgical jig so that said guide axis is perpendicular to said target axis; and
verifying a correct positioning of said surgical jig by the presence of an image of at least a part of said first region.

19. The method of claim 18, wherein said verifying step is accomplished by identifying the presence of said image of at least part of said first region using fluoroscopy.

20. The method of claim 19, wherein said verifying step is accomplished when said image of at least a part of said first region at least partly surrounds an image of said target.
